(19) United States
(12) Patent Application Publication
(10) Pub. No.: US 2013/0066134 A1
(43) Pub. Date: Mar. 14, 2013

(54) Multiplexed Radiation Therapy

(71) Applicant: Mark Carol, Burlingame, CA (US)

(72) Inventor: Mark Carol, Burlingame, CA (US)

(21) Appl. No.: 13/567,047

(22) Filed: Aug. 4, 2012

Related U.S. Application Data
(60) Provisional application No. 61/524,130, filed on Aug. 16, 2011.

(51) Int. Cl. A61B 6/04; A61N 5/10

(52) U.S. Cl. 600/1; 378/65; 378/62

(57) ABSTRACT

A radiation system is provided comprising a single source of therapeutic radiation that is directed toward at least two treatment spaces in succession. Each treatment space houses a patient to be treated, each patient being secured generally by a separate supporting device adapted to support the patient in a generally sitting or standing or horizontal position. The x-ray source first delivers at least one beam to the first patient in the first space and then the x-ray source/patient relationship is adjusted so as to deliver an at least one beam to an at least second patient in a second space.

### CROSS-REFERENCE TO RELATED APPLICATIONS

This application is entitled to the benefit of Provisional Patent Application 61/524,130 filed Aug. 16, 2011.

### BACKGROUND

1. Field of Invention

This invention relates to the delivery of radiation therapy, specifically to delivering multiplexed radiation treatments whereby a single radiation generating device or the beam from a single device is used to treat patients in more than one room in succession.

Typically in radiation therapy each room that contains a radiation generating device is used to treat patients only in that room. A treatment consists of positioning a patient on a treatment couch and then maintaining a constant patient position while a radiation therapy treatment beam is rotated or positioned about the patient surface so that the radiation therapy beam enters the patient from a range of orientations.

In the current practice of radiation therapy the patient typically lies down on a couch and is treated either in the prone or supine position, even though most patients are ambulatory. Often times, in order to allow the radiation beam to enter through different regions of the skin while it is rotated about the patient, the couch may itself be moved to a different orientation relative to the treatment beam. This requires a significant amount of floor space in order to accomplish.

Radiation therapy rooms must be designed and built to withstand the impact of the radiation used to treat patients. The walls surrounding the treatment room are shielded to block passage of photons or x-rays that pass through the patient, thereby protecting medical personnel and other individuals who might be in the vicinity of the treatment room. Shielded walls are either considered primary or secondary walls. Primary walls are those that would be impacted directly by the treatment beam if there was no patient present. Secondary walls are those that are impacted by x-rays after they bounce off the patient, another wall, or pieces of equipment in the room. Primary walls may be as much as 2 or 3 times as thick as secondary walls, often consisting of as much as seven or eight feet of concrete. In conventional radiation therapy, as the treatment beam rotates about the patient, it can sequentially points at a sidewall, the floor, the other sidewall, and the ceiling. This means that portions of these four walls must be treated as primary shielding and must be as much as seven or eight feet thick, requiring additional space for the treatment vault.

As a result of these and other reasons, radiation therapy can be considered to be essentially inefficient in terms of utilization of equipment, of space, and of capital resources. It is inefficient in terms of utilization of expensive equipment because the beam created by the accelerator, the most expensive piece of equipment involved in the treatment, is used only a small percentage of the time the patient is in room. Most of the “in room” time is spent setting the patient up for treatment and taking them off the table and out of the room after the treatment is over.

It is inefficient in terms of space because it requires a room large enough to rotate a patient, who is lying down on table, and the gantry, about an isocenter. It also requires a large amount of space due to the need for significant amounts of shielding.

Due to the need to provide each treatment room with expensive equipment installed in a large amount of space that is used only a small portion of the time and with each piece of equipment requiring its own operating and support personnel, the delivery of radiation therapy is expensive both in terms of capital and personnel. Proton therapy devices address the efficiency of equipment utilization by multiplex the treatment beam so that several patients can be treated at one time. But due to the need for extremely large gantries used to rotate the beam about the patient, they remain inefficient in terms of space and cost.

Current treatment is also inefficient in terms of the use of advanced imaging technologies such as CT, MRI, and PET. Because the radiation is delivered using a gantry that rotates about the patient, it is difficult to implement an imaging gantry that also rotates about the patient. As a result modern treatments are not as precise as they could be.

Many of these issues can be traced to the history of the field. In the early days of RT and tomographic imaging, the equipment was very time consuming to use, requiring up to an hour to image or treat a patient. Therefore, the supine position was used in order to keep patients comfortable. Now, CT images can be generated in seconds, MRI images in minutes, and radiation treatments delivered with less than 10 minutes of in-room time. In addition, the vast majority of patients are imaged electively, and the vast majority of radiation therapy patients walk into the treatment room under their power.

Therefore it is has become desirable to provide a radiation therapy method and system that is efficient in terms of utilization of equipment and space. It is also desirable to provide a method that is cost efficient and that allows concurrent advanced modality imaging of patients prior to and during their treatment.

2. Description of Prior Art

U.S. Pat. No. 5,349,198 teaches a beam generation device for particles whereby a plurality of beam utilization rooms are disposed around the rotational axis of a rotatable deflection electromagnet at a predetermined distance from a beam supply device for supplying a particle or radiation beam to a therapy or experiment equipment. U.S. Pat. No. 5,433,693 teaches patient treatment rooms circularly spaced around a nuclear reactor core for the purposes of delivering fast neutrons to treat cancer. U.S. Pat. No. 7,531,818 teaches a radiation system comprising an eccentric gantry arranged in connection with multiple treatment rooms separated by radiation-shielding separating members each with the capability to have simulation performed while a patient is being treated in another room. U.S. Pat. No. 7,796,730 discloses an irradiation treatment apparatus designed to treat patients in the standing position whereby the patient rotates in front of the treatment beam and where a CT scanner that drops down from the ceiling and generates images without rotating the patient is used to localize targets prior to treatment. 20100090122 teaches a multi-field irradiation charged particle cancer therapy method and apparatus using a fixed orientation synchrotron source relative to a rotating patient to yield tumor irradiation from multiple directions but in a single room only.

Each of these patents teaches the use of protons or other magnetically charged ions for the purposes of treatment and use technologies for directing these particles that involve the use of magnets and magnetic fields to direct the radiation beam. None of these patents disclose how to treat patients with photons, a particle that cannot be deflected by a magnet field, except by first using electromagnetically deflected proton or deuteron and electron beams to generate scanned neutral beams (U.S. Pat. No. 7,531,818), and none teach teaching a vertical patient that is rotated in front of the beam. Furthermore, U.S. Pat. No. 7,531,818 teaches also the use of a simulation system to set up the patient by a means that requires the imaging system to be in-line with the treatment beam and thus must be moved out of the way of the treatment beam when it is in use or into another treatment room entirely in order to simulate in the second room; it is not possible to simulate and treat multiple fields nor does it teach the use of an orthogonal imaging system. U.S. Pat. No. 7,531,818 also does not teach how to treat patients with multiple fixed fields or with a rotational arc in either a single or multiple rooms.

20100090122 teaches the use of a fixed orientation proton source relative to a rotating patient in a semi-vertical, sitting, or laying patient positioning to yield tumor irradiation from multiple directions but only in a single room and not with the beam on continuously. U.S. Pat. No. 7,796,730 discloses an irradiation treatment apparatus designed to treat patients in the standing position whereby the patient rotates in front of the treatment beam, thereby delivering multiple fields or even a rotational arc, and where a CT scanner is used to localize targets prior to treatment. However, it references specifically and only protons or hadron particles and never mentions or references the possibility of using photons to treat. It also does not teach multiplexing of the treatment beam or multiple room/patient deployment in front of a single treatment beam. U.S. Pat. No. 7,603,164 teaches the combined use of a CT and linear accelerator where the CT is separate from the linear accelerator requiring a common bed that is moved between the linac and the CT scanner. It teaches only the recumbent position with a rotating linac and rotating CT scanner.

Bagshaw and colleagues (A versatile radiotherapy treatment chair By C. J. Karzmark, Bagshaw, M. D., Huisman, P. A. D. Engr. and Joyce Lawson. BJR 53: 636: 1190-1194, 1980) described in 1980 a novel treatment setup that had been used for 10 years consisting of a horizontally deployed linear accelerator emitting photons to treat a patient positioned in a chair. Although this system treated vertical patients rotationally, it did not include imaging and had no ability to deliver treatments to multiple rooms or setups.

U.S. Pat. No. 7,603,164 teaches the use of a C-arm in conjunction with a linear accelerator, but the C-Arm is used to house imaging components and not the accelerator which is housed in a typical gantry configuration. U.S. Pat. No. 7,729,473 teaches a c-arm based system in conjunction with cobalt radiation sources, the c-arm being used solely for imaging. U.S. Pat. No. 6,968,035 teaches the use of a linear accelerator mounted on a c-arm. In this application the C-arm is used to rotate the source about the patient much as is accomplished by a gantry; it is not used to move the treatment device in/out of the treatment space. U.S. Pat. No. 6,888,919 references the use of an adjustable length c-arm to deliver imaging equipment into the field of view to be targeted by a radiation therapy beam.

U.S. Pat. Nos. 5,418,372 and 5,321,271 teach a therapy device mounted on a positioning means such as a C-arm to direct an electron beam to the desired site on the patient. A true c-arm with appropriate degrees of freedom is taught but the scope is limited to an electron beam used to deliver intraoperative radiation therapy. In addition, the C-arm is not used to move the accelerator in and out of the treatment space, rather wheels on the c-arm are used to meet this need.

Therefore there is a need to develop a radiation therapy system that provides the ability to deliver radiation therapy, and especially photon-based radiation therapy, to patients in the standing, sitting, and horizontal positions, using a single source of therapeutic radiation multiplexed so as to deliver a series of fixed or rotational fields in succession to a single patient support platform in each of multiple treatment rooms, or to a succession of patient support platforms sequentially deployed in a single room, each treatment performed with concurrent imaging.

### SUMMARY

A radiation system is provided comprising a single therapeutic radiation source that is directed toward at least two patients in succession. Each patient is secured generally by a separate supporting device adapted to support the patient in a generally sitting or standing or horizontal position. The radiation source first delivers at least one beam to the first patient and then the radiation source/patient relationship is adjusted so as to deliver an at least one beam to an at least second patient.

In some embodiments the therapeutic radiation source is mounted on a turntable so that it can be rotated so as to be directed toward more than one treatment room in succession. In another embodiment the radiation source can be translated in the horizontal plane so as to be directed toward more than one treatment room in succession. In a further embodiment at least two patients in succession are brought in front of the single radiation source by translating the patients along a linear track or by rotating the patients by a carousel-type mechanism.

In a preferred embodiment the therapeutic radiation source can be translated in a vertical direction in order to treat targets at different heights and also can be angled in the vertical plane to deliver beams from different angles. In another embodiment the radiation source can be mounted on a curved rail system so as to move in the vertically plane around an isocenter allowing it to be used to treat patients who are horizontal. In a further embodiment the radiation source is mounted on a robot that can reposition the beam to treat in different rooms and patients in different positions.

In some embodiments the therapeutic radiation source is self-shielded, in some embodiments the physical space enclosing the radiation source is shielded such that the shielding rotates or translates with the radiation source as it adjusts to treat different rooms.

In some embodiments, the therapeutic radiation source includes a beam adjuster that is used to adjust the size and shape of the beam, the beam adjuster rotating and translating with the radiation source. Alternatively, the beam adjuster can be placed in the wall separating the radiation source from the patient.

In some embodiments the patient and its supporting device, mounted on top of a turntable, can be translated vertically, horizontally, or angled.

In some embodiments provision is made for an imaging system, associated with each target turntable, deployed orthogonal to the therapeutic radiation beam and intersecting the target. The imaging system may be x-ray based, a MRI, PET, and any other means for generating a 2-D or 3-D image or dataset of the patient. In a preferred embodiment the imaging source and the imaging detector are mounted in the walls enclosing the patient turntable.

In some embodiments provision is made for a megavoltage detector panel that is mounted in the path of the therapeutic radiation beam after the therapeutic radiation beam passes through the target. In the preferred embodiment the megavoltage imaging panel is mounted in the wall of the space enclosing the patient turntable that is directly opposite the therapeutic radiation source.

In a further embodiment, the treatment beam itself is switched between rooms rather than changing the position of the physical hardware used to generate the beam.

In one aspect, a method of irradiating a target in at least one patient is provided. The method comprises the steps of positioning the patient in a supporting device that accommodates a roughly sitting or standing position and that is mounted on a turntable, adjusting the x-y-z and angular position of the patient so that the target is at the center of the turntable, irradiating the patient with a therapeutic beam of radiation that is held fixed in space while the patient is rotated continuously or rotating the patient in steps to additional positions so as to deliver the radiation from a plurality of positions distributed around the patient, the parameters of radiation, such as the size and shape of the radiation field and the duration and amount of radiation delivered from any given position of the patient, being adjusted and with imaging of the patient able to be performed prior to and during the time that radiation beam is delivered to insure that the treatment is being delivered correctly.

Another provided method comprises the steps positioning a first patient in a supporting device that accommodates a roughly sitting or standing position and that is mounted on a turntable, adjusting the x-y-z position of the patient so that the target is at the center of the turntable, and then irradiating the patient with an therapeutic beam of radiation that is held fixed in space, rotating the patient continuously, or the patient is rotated in steps to additional positions, so as to deliver the radiation from a plurality of positions distributed around the patient, the parameters of radiation, such as the size and shape of the radiation field and the duration and amount of radiation delivered to any given position of the patient, being adjusted to fit the patient, imaging of the patient being performed prior to and during the time that radiation beam is delivered to insure that the treatment is being delivered correctly, with a first patient being removed from the supporting device while the radiation beam or radiation beam generating device is rotated or translated to point to an at least second patient who has been positioned for treatment in an at least second treatment room.

A further provided method comprises the steps positioning a first patient in a supporting device that accommodates a roughly sitting or standing or recumbent position and that is mounted on a turntable, adjusting the x-y-z position of the patient so that the target is at the center of the turntable, then rotating a radiation beam mounted on a vertically oriented curved track such that the therapeutic radiation beam can be brought to bear on the patient, changing the degree of angulation of the radiation beam and the patient simultaneously or separately, so as to deliver the radiation from a plurality of positions distributed around the patient, with the parameters of radiation, such as the size and shape of the radiation field and the duration and amount of radiation delivered to any given position of the patient, able to be adjusted and with imaging of the patient able to be performed prior to and during the time that radiation beam is delivered to insure that the treatment is being delivered correctly.

A further provided method comprises the steps positioning a first patient in a supporting device that accommodates a roughly sitting or standing or recumbent position and that is mounted on a turntable, adjusting the x-y-z position of the patient so that the target is at the center of the turntable, then rotating a therapeutic radiation beam mounted on a vertically curved track such that the radiation beam can be brought to bear on the patient, changing the degree of angulation of the radiation beam and the patient simultaneously or separately, so as to deliver the radiation from a plurality of positions distributed around the patient, with the parameters of radiation, such as the size and shape of the radiation field and the duration and amount of radiation delivered to any given position of the patient, able to be adjusted and with imaging of the patient able to be performed prior to and during the time that radiation beam is delivered to insure that the treatment is being delivered correctly, with a first patient being removed from the supporting device while the radiation beam or radiation beam generating device is rotated or translated to point to an at least second patient who has been positioned for treatment in an at least second treatment room.

### DRAWINGS

FIGS. 1a & b are side perspectives of a two-room treatment facility where (a) treatment beam is directed toward a first patient and (b) where treatment beam is directed toward a second patient.

FIGS. 2a & 2b are top perspectives of a two-room treatment facility where (a) treatment beam is directed toward a first patient and (b) where treatment beam is directed toward a second patient.

FIG. 3 is a top perspective of a four-room treatment facility laid out in a rotary fashion

FIG. 4 is a top perspective of a four-room treatment facility laid out in a linear fashion

FIG. 5 is a top perspective of an eight-room treatment facility laid out in a double linear fashion

FIG. 6a-6d are top perspectives of a four-room treatment facility laid out in a carousel fashion

FIG. 7a-6b are top perspectives of a four-room treatment facility laid out in a carousel turnstile fashion

FIG. 8a-8c are side perspectives of a two-room facility equipped with the ability to treat recumbent patients on one room where (a) the treatment beam is contained in the central housing, (b) the treatment beam on its curved rail support are brought into a treatment room to treat a recumbent patient and (2) where the treatment beam has been rotated to treat a standing patient in a second room

FIG. 9 is a top perspective of a three-room facility with the ability to treat recumbent patients in one room where (a) is the patient oriented one direction and (b) patient oriented the other direction

FIG. 10 is a side perspective of a two-room facility with a robot used to deploy the treatment where (a) the robot and treatment beam are contained in the central housing, (b) where the robot has deployed the treatment beam into one treatment room, (c) where the robot has rotated to (d) be deployed into a second treatment room

FIG. 11 is a side perspective of a two-room facility where the treatment beam itself is redirected rather than rotating the beam generation device.

### DETAILED DESCRIPTION OF VARIOUS EMBODIMENTS

Various embodiments of the present invention are described hereinafter with reference to the figures. It should be noted that the figures are not drawn to scale and elements of similar structures or functions are represented by like reference numerals throughout the figures. It should also be noted that the figures are not intended to facilitate the description of specific embodiments of the invention. They are not intended as an exhaustive description of the invention or as a limitation on the scope of the invention. In addition an aspect described in conjunction with a particular embodiment of the present invention is not necessarily limited to that embodiment and can be practiced in any other embodiments of the present invention. It will be appreciated that while various embodiments of the invention are described in connection with radiation treatment of tumors, the claimed invention has application in other industries and to targets other than cancers.

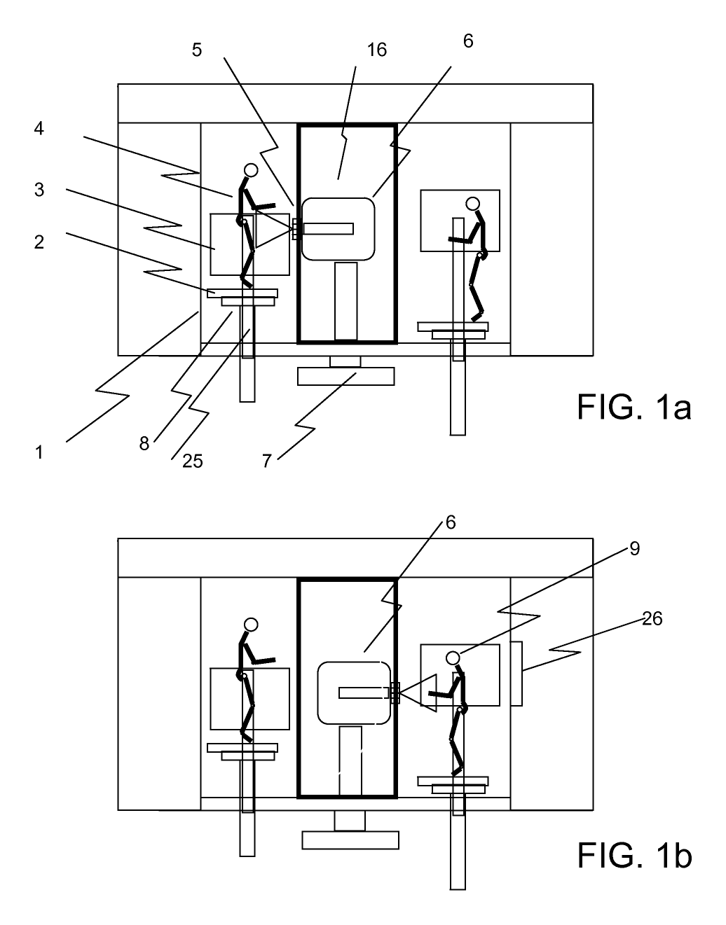

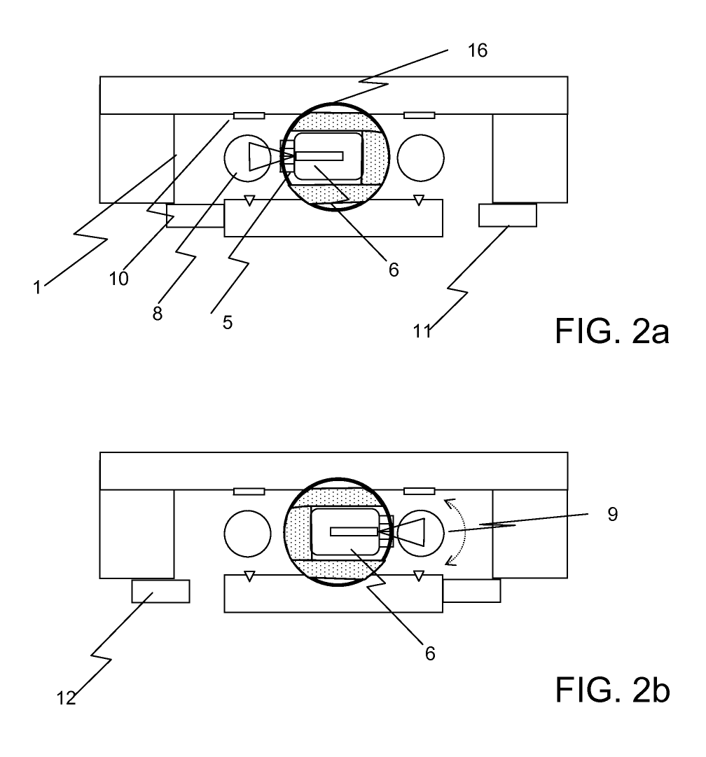

In a preferred embodiment of the present invention as depicted in FIG. 1a and FIG. 2a, each treatment system consists of one or more patient treatment rooms with shielding (1) surrounding a centralized space (16) containing a single source of radiation (6) where the single source of radiation is used to deliver a beam to each of the treatment rooms that surrounds it. The source of radiation can be any device that generates a radiation beam that can be used for therapeutic purposes. By way of example, in some embodiments any radiation source can be used where the beam generating device and the beam itself can be deployed in its entirety within a contained space surrounded by treatment rooms such as a megavoltage linear accelerator or a cobalt x-ray source. Alternatively, the radiation source may be proton beam, or heavy ion beam where the beam generating device may be located remotely but the beam itself can be deployed within a contained space surrounded by treatment rooms.

Regardless of the source of the beam, the beam generating device or the beam itself may be mounted on a device that allows it to be raised or lowered (25). The device upon which the beam generating device or the beam itself also can be equipped with means to angle it up or down relative to the horizontal. The device supporting the beam or beam generating device can be secured to a means for rotating the beam (7) or the beam generating device around the vertical axis 360 degrees. The mechanisms for providing vertical, angular, and/or rotational motion are well known to those skilled in the art and therefore they are not described in great detail in order to simplify the description of the invention.

A beam adjuster or collimation device may be interposed between the beam source and the treatment room (5). The collimation system may be secured to the end of the beam generating device, so that it moves with the device, or in the wall through which the beam passes. In a preferred embodiment the collimation system is mounted to the beam generating device and consists of a multileaf collimator. In other embodiments the multileaf collimator may be replaced by any other form of collimation system used typically in radiation therapy. If the collimation device is interposed between the beam generating device and the treatment room such as in the wall, a separate collimation system is deployed for each treatment room.

Each treatment room consists of a platform, which may include a patient support assembly, mounted on an table (2), with the ability to have its position adjusted in anywhere from 2 to 6 degrees of freedom, that is secured to a turntable (8) that is in turn secured to the floor. The patient support system can be used to maintain the patient in a sitting or standing or semi-standing position. The table allows the patient support assembly to be moved in any or all combinations of directions: a lateral direction (y direction or side to side), a longitudinal direction (x direction or closer-farther from the radiation beam), angled forward/backward and side-to-side, or raised/lowered. The table and/or the patient support system can be moveable separately in a vertical direction. The motions of the table and supporting device in combination allows up to six degrees of freedom and a wide range of positions of the patient including the ability to move the target located inside the patient to the center of rotation of the turntable. The turntable is used to rotate the patient relative to the treatment beam. The mechanisms for providing translational and rotational motion are well known to those skilled in the art and therefore they are not described in great detail in order to simplify the description of the invention. In general, any mechanisms may be used to provide translational or rotational motion.

Each room also may contain imaging technology or other technology (3) used to localize the patient relative to the treatment room and the radiation beam. The preferred embodiment includes an imaging system that is mounted in the walls or adjacent to the walls of each chamber housing the patient turntable such that the imaginary line connecting the two sides of the imaging system passes across the vertical space occupied by the turntable orthogonal to the direction of the treatment beam.

There are various imaging systems that can be deployed in this manner. These include KV imaging consisting of a KV x-ray source and an imaging detector panel or array (10). The KV imaging system can be used to image the patient before and during treatment thereby determining the location of the target, or markers placed within the target.

MV imaging also can be used to determine the location of the target inside the patient. A MV imaging plate can be mounted in a third wall as in FIG. 1b (26) of the enclosure such that it is intersected by the treatment beam after it passes through the patient (26).

Magnetic resonance imaging also may be used to image the patient. In this approach large imaging magnets are installed in place of the KV beam source and detector system. The two magnets generate a magnetic field between them allowing patients or portions of patients introduced into the field to be imaged. In MR imaging the patient need not rotate in between the MR imaging plates since MR is in and of itself a volumetric imaging modality. However, for MR imaging to be accurate and precise, the magnets must be as close to the patient as possible. This can be achieved by mounting each of the pair of MR magnets on moving tracks such that they can slide toward the patient for imaging, then slide back away from the patient for treatment such that the patient support structure is able to rotate freely during treatment.

Positron emission tomography also may be used to image a patient. Positron coincidence detectors are mount in place of the KV x-ray source and detectors or the MRI magnets. These detector panels can be used to generate information about the metabolic activity of the target thereby allowing proper radiation dosing.

It may also possible and at desirable to multiplex the imaging beam if such is used. For instance, when using plain imaging or CT scanning, rather than placing complete, hardware including x-ray source and imaging array in each room, it would be necessary only to place an x-ray source in each room. The imaging plate would be mounted on the turntable such that it was pointing at a second patient while the therapy x-ray source was pointing at a first. The second patient can be imaged for treatment while the first patient is being treated and so forth.

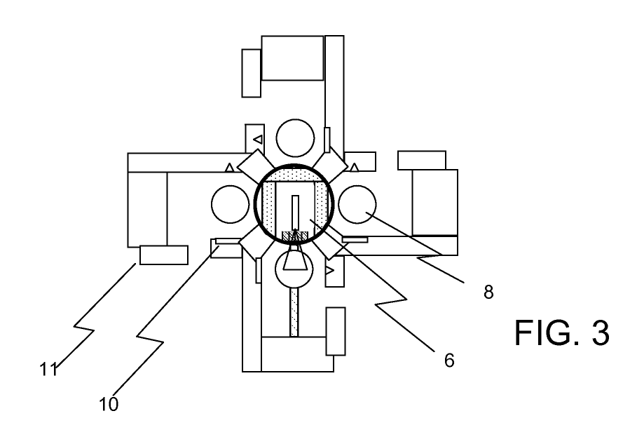

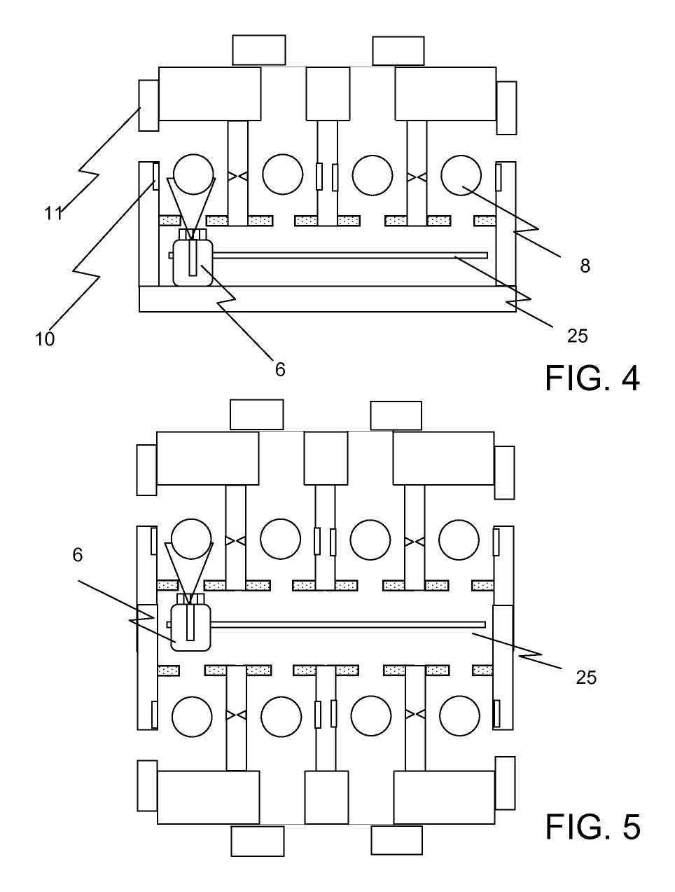

Multiple rooms are laid out in a circumferential pattern around a center chamber housing the treatment beam. The treatment beam can be rotated to point at each room in turn. In the preferred embodiment there are four treatment rooms (FIG. 3) laid out in such a manner although two and three room and more than four room deployments are possible as well. The center chamber housing the treatment beam has shielding in place to protect the rooms at which the beam is not pointing from being exposed to radiation. This shielding can be designed to rotate with the radiation beam or can be fixed in place with doors that open into each room when the beam is pointed into that room.

In operation, as shown in FIG. 1a,b and FIG. 2a,b, a patient in a first room (1) is positioned on a platform and may be immobilized in a support structure in either an essentially sitting or standing position. Using means commonly employed in the delivery of radiation therapy to determine the location of the target within the patient, the target inside the patient relative to the turntable is adjusted by moving the table (2). The means for determining target location can include the use of a KV imaging system, a MV imaging system, MRI, PET or a nonradiation imaging system such as radio or magnetic beacons, skin tattoos, and other devices affixed to or within the patient. Once the patient position has been adjusted so that the target is correctly positioned at the center of rotation of the patient turntable (8), the patient can be treated. Treatment can be delivered by a continuous rotation of the patient or in a series of individual fields spaced about the patient. In a continuous rotation, the treatment beam is kept on while the patient turntable makes a rotation. For delivering individual fields the patient is rotated a predetermined amount to the first field, the treatment beam is turned on for a predetermined amount of time and then turned off, the patient is rotated by a second predetermined amount to the second field, the treatment beam is turned on again for a predetermined amount and then turned off. This process repeats until all the fields are delivered.

Once the treatment has been concluded, the patient is removed from the support fixture, if one was used, and exits the room through a moveable door (12). In a preferred embodiment where there are multiple treatment rooms being serviced by the single radiation beam, the beam is rotated so that it is oriented toward the next treatment room (9) where a second patient has entered through a second moveable door (11) and has been positioned correctly and the process as described above repeated:

In various embodiments biplanar x-ray images can be used to determine location of implanted markers or radio-opaque regions of anatomy within the patient. With the patient present on the platform, a first KV or MV image is generated of the patient. The turntable is then rotated 90 degrees and a second image is generated. These two images can be used to identify the location of the target to be treated using means commonly known in the field. Alternatively, KV imaging systems that are able to generate data with respect to depth from a single angle, such as a scanning digital x-ray system using an inverse imaging geometry, can be used. In this approach, only a single image is required to determine the position of a marker or radio-opaque anatomy within the patient because the imaging system provides positional information in the z-direction. This allows also the position of the markers to be tracked during treatment. Such a system also can be used to generate real time CT images. In this approach the patient is scanned continuously as the patient is rotated for therapy. The stored data from an initial scan acquired during a complete rotation of the patient is updated continuously while the patient is being treated and simultaneously imaged in order to give real time information on the position of the patient and structures within the patient.

In another embodiment, FIGS. 4 & 5, the treatment beam is translated between rooms rather than being rotated between rooms. The support device for the treatment beam is installed in a linear track (24) that runs alongside or between a single or double line of treatment rooms placed linearly one next to each other. After treating a patient in a first room the treatment beam (6) is translated along the track to be brought to bear on a next room where a second patient is waiting to be treated. The beam can be brought to bear on all rooms in succession or in any other sequence of rooms.

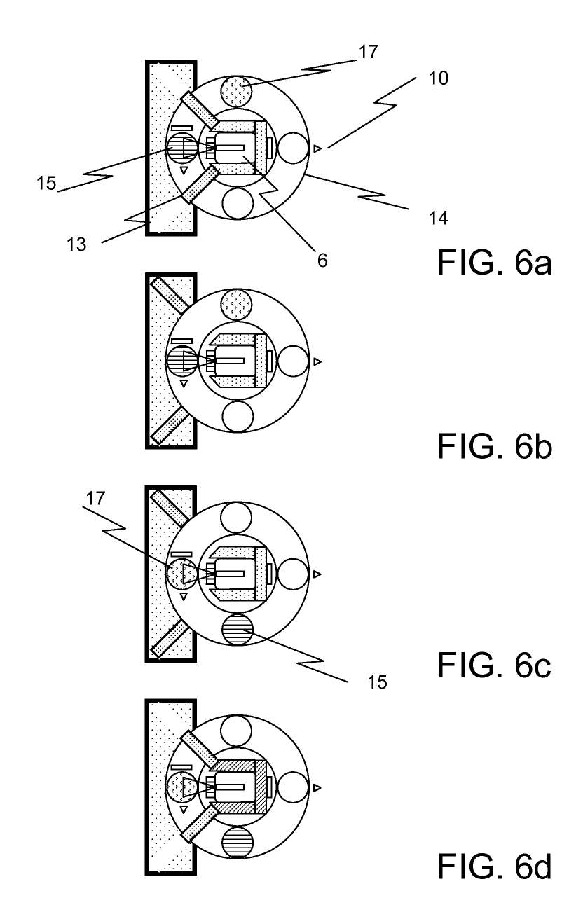

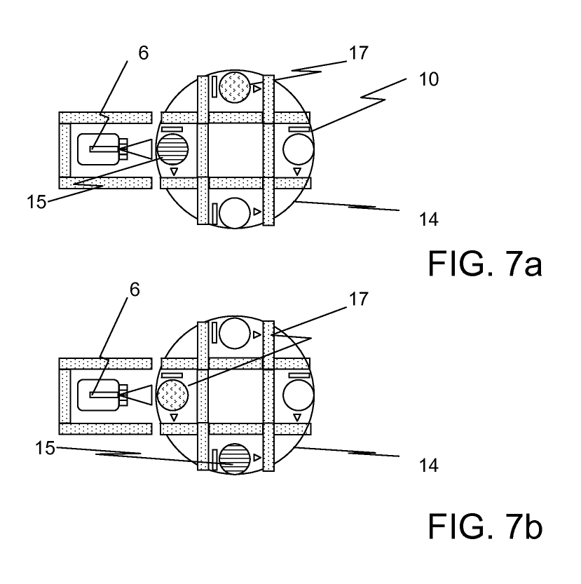

In another embodiment, the patients are moved to be in front of the treatment beam rather than the treatment beam being moved to treat different patients. In this embodiment the treatment beam does not move, rather the platforms on which the patients are positioned move. In one embodiment, FIG. 6, the patient support platforms are mounted on a large rotary disc or carousel (14) with the treatment beam housed in the center of the disc pointing in a fixed direction. After a first patient (15) is treated, the entire disc or carousel rotates around the treatment beam housing so as to bring a second patient (17) in opposition to the treatment beam. Doors (13) open and close on the physical space in which the platform sits while receiving treatment so as to allow a first support platform to move out of, and a second support platform to move into, the treatment space. An imaging system can be mounted within the treatment space as well as outside the treatment space (10). In an alternate embodiment the patient support platforms can be mounted on a linear track or belt that brings the patients in turn in front of the treatment beam. In further embodiment FIG. 7, the patient support platforms are mounted on a large rotary disc or carousel (14) with the treatment beam housed outside of the disc pointing in a fixed direction toward the center of the disc. After a first patient (15) is treated, the entire disc or carousel rotates around the treatment beam housing so as to bring a second patient (17) in opposition to the treatment beam. There are no doors to open and close, and each patient space can be equipped with its own imager (10).

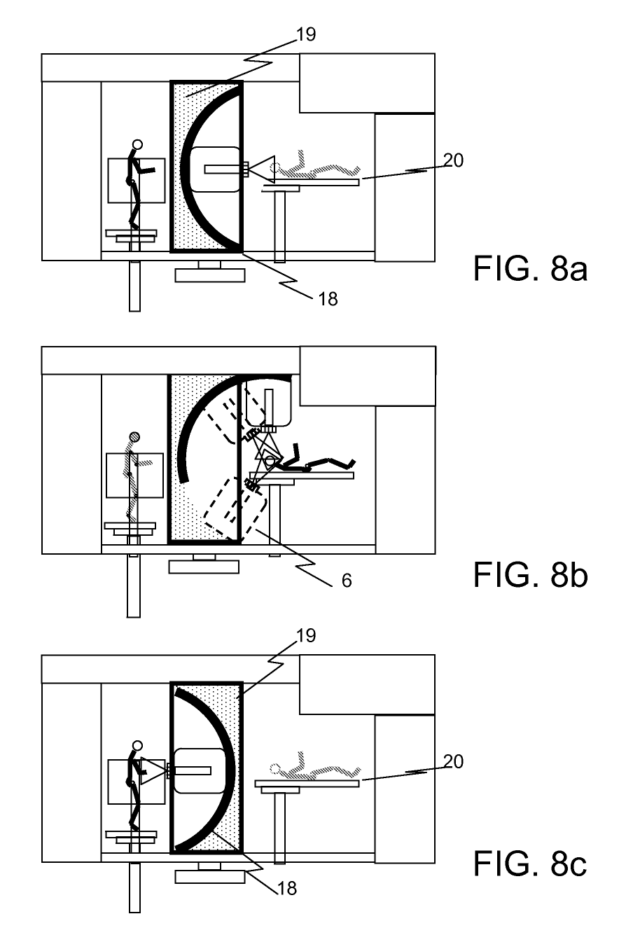

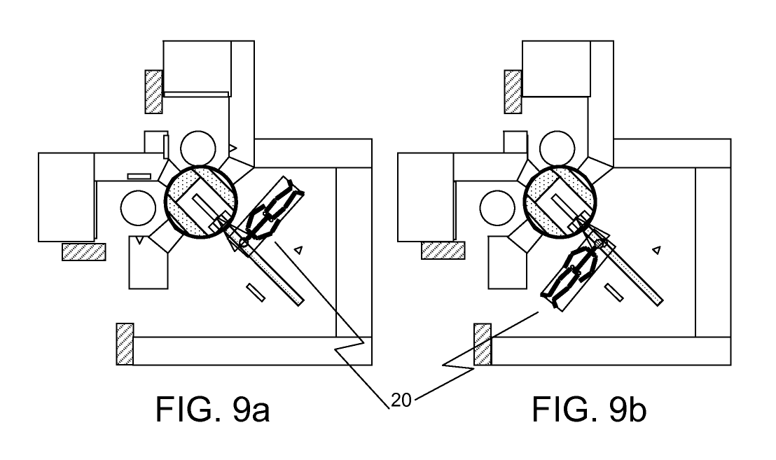

It also may be desirable to use a multiplexed approach to treat patients that need to remain in the recumbent position either because of age, infirmary, or the duration of the treatment. It may also be desirable to deliver the beam from angles that cannot be achieved by a fixed horizontal beam, such as a superior/inferior beam direction for treating breast cancer in a sitting or standing patient. In a further embodiment (FIG. 8) the radiation beam (6) can be mounted on a curved rail (18) that is contained within the central compartment housing the treatment beam (6) and a support rail for the curved rail (19). The curved rail and beam can be rotated along its curvature such that it is brought out of the central compartment (FIG. 8b) coming to lie within the treatment room. Various beam angles can be achieved by adjusting the degree of rotation of the curved rail and the treatment beam on the curved rail (FIG. 8c), as well as by changing the angular position of the patient support structure. In this embodiment that patient support structure may include a multi-degree of freedom couch (20), in place of the multi-degree of freedom table, on which the patient can be positioned, or it may include a standing or sitting fixture. Once the treatment of a patient is finished the curved rail with attached treatment beam can be retracted back into the central compartment so that the beam can be brought to bear on an at least second patient in succession by any of the means described above. This approach can be used to deliver a series of fixed fields to the patient or a rotational treatment. However, unlike the rotational treatment delivered to a vertical patient, that can be delivered in a single uninterrupted arc of an appropriate amount, the arc delivered using this embodiment must be done in sections. With the treatment couch (20) oriented in one direction (FIG. 9a), the treatment beam and curved rail can be rotated from a completely inferior orientation, aiming up at the couch, through approximately 180 degrees to a completely superior orientation, aiming down at the couch. This will deliver radiation from one side of the patient only. The treatment couch can then be rotated 180 degrees (FIG. 9b) so as to point in the completely opposite direction, at which time the treatment beam and curved rail can be rotated from a completely superior orientation, aiming down at the couch, to a completely inferior orientation, aiming up at the couch, thereby delivering radiation from the other side of the patient. It should be understood that individual arc segments of less than 180 degrees may be used to deliver treatment to a horizontal patient.

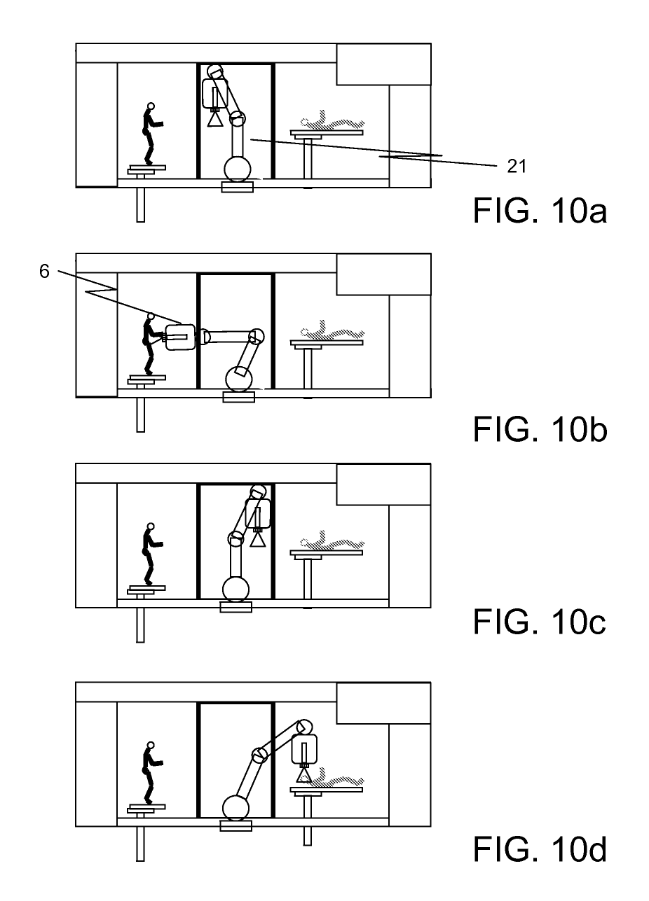

In another embodiment (FIG. 10) the treatment beam (6) can be mounted on a multiple degree of freedom robot (21) that is housed in the central compartment. The robot can be rotated or translated to point toward the active treatment room and then the x-ray source can be deployed into that room using the arm on the robot. The robot can point the beam in a general horizontal direction to treat patients that are standing and who are rotated in front of the beam. The beam also can be oriented in a generally vertical direction (pointing down or up or any orientation in between) to treat patients that are lying down, with the robot changing the orientation of the beam about the patient to deliver a range of beam directions.

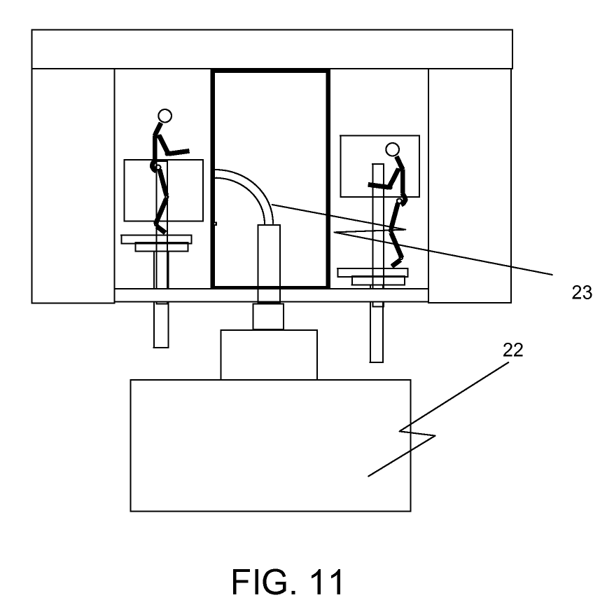

In a further embodiment (FIG. 11) the treatment beam itself can be redirected to deliver radiation to at least two treatment rooms in succession. This can utilize a beam generation device for particles or photons (22). A plurality of beam utilization rooms can be disposed around the rotational axis of a rotatable deflection electromagnet (23) such that the treatment beam can be deflected in turn into each room where a vertically oriented patient is rotated in front of the beam with orthogonal imaging provided for verifying patient position throughout the course of treatment. The source of the treatment beam can be under the treatment rooms or in some other remote location such that the beam can be brought into the central column containing the rotatable deflection electromagnet. The guide used to direct the treatment beam can be adjustable vertically in addition to be able to rotate around the central axis of the beam.

I claim:

1. An apparatus for performing radiation therapy, said apparatus comprising:
a. A means for generating a beam of radiation
b. Means for defining at least two treatment spaces such that the at least two treatment spaces are shielded from each other
c. Means for supporting in each treatment space &patient in a generally sitting or standing or horizontal position
d. A means for adjusting the orientation of the beam of ionizing radiation relative to the at least two patients so that the beam is first directed toward a first patient in a first treatment space and the beam is then directed toward the at least second patient in a second treatment space,
whereby the at least two patients in separate treatment spaces receive radiation therapy treatments in succession from a means of generating beams of ionizing radiation with each being shielded from the treatment delivered to the at least one other patient.

2. The apparatus of claim 1 wherein said means for generating comprises a radioactive source or an x-ray generating device.

3. The apparatus of claim 1 wherein said means for generating comprises a proton accelerator or heavy particle accelerator.

4. The apparatus of claim 1 wherein said means for generating can be translated in a vertical plane.

5. The apparatus of claim 1 wherein said means for generating includes a means for translating the beam of radiation in a vertical plane.

6. The apparatus of claim 1 wherein said means for generating can be angled in a vertical plane.

7. The apparatus of claim 1 wherein said means for generating includes a means for angling the beam of radiation in a vertical plane.

8. The apparatus of claim 1 wherein said means for generating includes a means for guiding and moving said means for generating along an arc in the vertical plane such that multiple beams can be delivered distributed around an isocenter located inside an at least one treatment space.

9. The apparatus of claim 1 wherein said means for generating includes a means for moving a beam of radiation along an arc in the vertical plane such that multiple beams can be delivered distributed around an isocenter located inside an at least one treatment space.

10. The apparatus of claim 1 wherein said means for generating includes a single means for adjusting the size and shape of the beam that is mounted to the single means for generating.

11. The apparatus of claim 1 wherein said means for generating includes a means for each treatment space for adjusting the size and shape of the beam when directed toward the treatment space.

12. The apparatus of claim 1 wherein said means for generating includes a means for shielding the means for generating from the at least two patient spaces.

13. The apparatus of claim 1 wherein said means for supporting can be secured to a device for rotating, translating, or angling the patient.

14. The apparatus of claim 1 wherein said means for adjusting comprises a rotatable turntable on which the means for generating is mounted such that the means for generating can be rotated in the horizontal plane so as to be directed toward at least two treatment spaces in succession.

15. The apparatus of claim 1 wherein said means for adjusting comprises a linear translation device on which the means for generating is mounted such that the means for generating can be translated in the horizontal plane so as to be directed toward at least two treatment spaces in succession.

16. The apparatus of claim 1 wherein said means for adjusting comprises a robot on which the means for generating is mounted such that the means for generating can be directed toward at least two treatment spaces in succession.

17. The apparatus of claim 1 wherein said means for adjusting comprises translation of the at least two treatment spaces in succession in front of the means for generating such that the first patient treatment space is translated into and then out of the path of the treatment beam and then the second patient treatment space is translated into and then out of the path of the treatment beam.

18. The apparatus of claim 1 wherein said means for adjusting comprises rotation of the at least two treatment spaces in succession in front of the means for generating such that the first patient treatment space is rotated into and then out of the path of the treatment beam and then the at least second patient treatment space is rotated into and then out of the path of the treatment beam.

19. The apparatus of claim 1 wherein said means for adjusting comprises electronic switching of the beam direction, without moving the means for generating, such that the treatment beam can be rotated in the horizontal plane so as to be directed toward at least two treatment spaces in succession.

20. An apparatus for performing radiation therapy, said apparatus comprising:
a. A means for generating a beam of radiation
b. Means for defining at least two treatment spaces such that the at least two treatment spaces are shielded from each other
c. Means for supporting in each treatment space a patient in a generally sitting or standing or horizontal position
d. A means for adjusting the orientation of the beam of ionizing radiation relative to the at least two patients so that at least one beam is first directed toward a first patient in a first treatment space and then at least one beam is directed toward the at least second patient in a second treatment space
e. A means for imaging the patient any point in the treatment process that is deployed in at least one treatment space
whereby the at least two patients in separate treatment spaces receive radiation therapy treatments in succession from a means of generating beams of ionizing radiation, with each being shielded from the treatment delivered to the at least one other patient, and with at least one patient imaged at some point during the treatment process in order to ascertain the location or shape or size of the patient or target and other internal structures and landmarks inside the patient.

21. The apparatus of claim 20 wherein said means for imaging is deployed in a fixed orthogonal relationship to the treatment beam.

22. The apparatus of claim 20 wherein said means for imaging may be x-ray based, a CT scanner a MRI scanner, a PET scanner, and any other means for generating a 2-D or 3-D image or dataset of the patient.

23. The apparatus of claim 20 wherein said means for imaging generates 3-D data by rotating the patient on a turntable in front of an imaging beam that itself does not rotate.

24. The apparatus of claim 20 wherein said means for generating comprises a radioactive source or an x-ray generating device.

25. The apparatus of claim 20 wherein said means for generating comprises a proton accelerator or heavy particle accelerator.

26. The apparatus of claim 20 wherein said means for generating can be translated in a vertical plane.

27. The apparatus of claim 20 wherein said means for generating includes a means for translating the beam of radiation in a vertical plane.

28. The apparatus of claim 20 wherein said means for generating can be angled in a vertical plane.

29. The apparatus of claim 20 wherein said means for generating includes a means for angling the beam of radiation in a vertical plane.

30. The apparatus of claim 20 wherein said means for generating includes a means for guiding and moving said means for generating along an arc in the vertical plane such that multiple beams can be delivered distributed around an isocenter located inside an at least one treatment space.

31. The apparatus of claim 20 wherein said means for generating includes a means for moving a beam of radiation along an arc in the vertical plane such that multiple beams can be delivered distributed around an isocenter located inside an at least one treatment space.

32. The apparatus of claim 20 wherein said means for generating includes a single means for adjusting the size and shape of the beam that is mounted to the single means for generating.

33. The apparatus of claim 20 wherein said means for generating includes a means for each treatment space for adjusting the size and shape of the beam when directed toward the treatment space.

34. The apparatus of claim 20 wherein said means for generating includes a means for shielding the means for generating from the at least two patient spaces.

35. The apparatus of claim 20 wherein said means for supporting can be secured to a device for rotating, translating, or angling the patient.

36. The apparatus of claim 20 wherein said means for adjusting comprises a rotatable turntable on which the means for generating is mounted such that the means for generating can be rotated in the horizontal plane so as to be directed toward at least two treatment spaces in succession.

37. The apparatus of claim 20 wherein said means for adjusting comprises a linear translation device on which the means for generating is mounted such that the means for generating can be translated in the horizontal plane so as to be directed toward at least two treatment spaces in succession.

38. The apparatus of claim 20 wherein said means for adjusting comprises a robot on which the means for generating is mounted such that the means for generating can be directed toward at least two treatment spaces in succession.

39. The apparatus of claim 20 wherein said means for adjusting comprises translation of the at least two treatment spaces in succession in front of the means for generating such that the first patient treatment space is translated into and then out of the path of the treatment beam and then the second patient treatment space is translated into and then out of the path of the treatment beam.

40. The apparatus of claim 20 wherein said means for adjusting comprises rotation of the at least two treatment spaces in succession in front of the means for generating such that the first patient treatment space is rotated into and then out of the path of the treatment beam and then the at least second patient treatment space is rotated into and then out of the path of the treatment beam.

41. The apparatus of claim 20 wherein said means for adjusting comprises electronic switching of the beam direction, without moving the means for generating, such that the treatment beam can be rotated in the horizontal plane so as to be directed toward at least two treatment spaces in succession

42. A method for delivering radiation to at least two treatment spaces in succession using a single radiation generating device, comprising the steps of:
a. Positioning a first patient in a first treatment space in a supporting device that can be translated x-y-z-angularly that accommodates the roughly sitting or standing or recumbent positions, the supporting device mounted on a turntable
b. Adjusting the x-y-z and angular position of the supporting device in the first treatment space so that the target inside or on the surface of the patient is at the desired location relative to the turntable
c. Directing a radiation beam from the radiation generating device into the first treatment space
d. Irradiating the first patient with the treatment beam of radiation, holding the beam fixed in orientation relative to the treatment space while the patient is rotated or translated relative to a vertical axis continuously or in steps to additional positions relative to the treatment beam such that the radiation beam can intersect the patient from a plurality of positions on the patient surface
e. Introducing a second patient into a second treatment space at any point before, during or after the treatment of the first patient
f. Adjusting the x-y-z and angular position of the first patient so that the target inside or on the surface of the patient is at the desired location relative to the turntable
g. Adjusting the position of the radiation generating device or the treatment beam relative to the treatment spaces so that the device or beam is directed toward the second treatment space
h. Irradiating the second patient with the treatment beam of radiation, holding the beam fixed in orientation while the patient is rotated or translated continuously or in steps to additional positions such that the radiation beam can be delivered from a plurality of positions distributed around the patient
Whereby a single means for delivering radiation can be used to treat in succession at least two patients in two treatment spaces.

43. The method of claim 40 furthermore comprising the step of adjusting the size and shape of the radiation beam and the duration and amount of radiation delivered from any beam position relative to the patient as each patient rotates in front of the treatment beam.

44. The method of claim 40 furthermore comprising the step of imaging of the patient in at least one treatment space whereby the position, shape, and size of the patient or the target inside the patient is ascertained

45. A method for delivering radiation to at least two treatment spaces in succession using a single radiation generating device, comprising the steps of:
a. Positioning a first patient in a first treatment space in a supporting device that can be translated x-y-z-angularly that accommodates the roughly sitting or standing or recumbent positions, the supporting device mounted on a turntable
b. Adjusting the x-y-z and angular position of the supporting device in the first treatment space so that the target inside or on the surface of the patient is at the desired location relative to the turntable
c. Directing a radiation beam from the radiation generating device into the first treatment space so as to irradiate the first patient with the treatment beam of radiation
d. Advancing the radiation generating device or the location of origin of the radiation beam as it is directed toward the patient such that the radiation beam can be delivered from a plurality of positions inside the treatment space
e. Rotating or translating the position of the patient relative to the treatment beam so that the radiation beam can intersect the patient from a plurality of positions on the patient surface
f. Introducing a second patient into a second treatment space at any point during or after the treatment of the first patient
g. Adjusting the x-y-z and angular position of the second patient so that the target inside or on the surface of the patient is at the desired location relative to the turntable
h. Adjusting the position of the radiation generating device or the treatment beam relative to the treatment spaces so that the device or beam is directed toward the second treatment space and can be used to irradiate the patient
i. Advancing the radiation beam or radiation generating device into the first treatment space such that the radiation beam can be delivered from a plurality of positions distributed around the patient
j. Rotating or translating the position of the patient relative to the treatment beam so that the radiation beam can be delivered from a plurality of positions distributed around the patient.
Whereby a single means for delivering radiation can be used to treat in succession at least two patients in two treatment spaces.

46. The method of claim 40 furthermore comprising the step of adjusting the size and shape of the radiation beam and the duration and amount of radiation delivered from any beam position relative to the patient as each patient rotates in front of the treatment beam.

47. The method of claim 40 furthermore comprising the step of imaging of the patient in at least one treatment space whereby the position, shape, and size of the patient or the target inside the patient is ascertained.
